SEQUENCE LISTING (12 sequences)

SEQ ID NO: 1 - protein, 38 aa, Artificial Sequence
  Synthetic peptide
  MOD_RES (2)..(2): PHOSPHORYLATION
DSHEKRHHGYRRKFHEKHHSHREFPFYGDYGSNYLYDN

SEQ ID NO: 2 - protein, 27 aa, Artificial Sequence
  Synthetic peptide
RKFHEKHHSHREFPFYGDYGSNYLYDN

SEQ ID NO: 3 - protein, 32 aa, Artificial Sequence
  Synthetic peptide
DSHAKRHHGYKRKFHEKHHSHRGYRSNYLYDN

SEQ ID NO: 4 - protein, 20 aa, Artificial Sequence
  Synthetic peptide
KFHEKHHSHRGYRSNYLYDN

SEQ ID NO: 5 - protein, 24 aa, Artificial Sequence
  Synthetic peptide
DSHAKRHHGYKRKFHEKHHSHRGY

SEQ ID NO: 6 - protein, 25 aa, Artificial Sequence
  Synthetic peptide
DSHAKRHHGYKRKFHEKHHSHRGYR

SEQ ID NO: 7 - protein, 13 aa, Artificial Sequence
  Synthetic peptide
RKFHEKHHSHRGY

SEQ ID NO: 8 - protein, 12 aa, Artificial Sequence
  Synthetic peptide
KFHEKHHSHRGY

SEQ ID NO: 9 - protein, 14 aa, Artificial Sequence
  Synthetic peptide
RKFHEKHHSHRGYR

SEQ ID NO: 10 - protein, 13 aa, Artificial Sequence
  Synthetic peptide
KFHEKHHSHRGYR

SEQ ID NO: 11 - protein, 8 aa, Artificial Sequence
  Synthetic peptide
KRHHGYKR

SEQ ID NO: 12 - protein, 7 aa, Artificial sequence
  Synthetic peptide
KRHHGYK

(19) United States
(12) Patent Application Publication
(10) Pub. No.: US 2021/0364534 A1
(43) Pub. Date: Nov. 25, 2021

(54) METHODS OF USING HISTATIN FOR DIAGNOSING AND TREATING A DRY EYE DISEASE OR OTHER OCULAR DISEASES

(71) Applicant: THE BOARD OF TRUSTEES OF THE UNIVERSITY OF ILLINOIS, Urbana, IL (US)

(72) Inventors: Vinay Kumar AAKALU, Chicago, IL (US); Sandeep JAIN, Oak Park, IL (US)

(21) Appl. No.: 17/286,671

(22) PCT Filed: Oct. 24, 2019
(86) PCT No.: PCT/US19/57807

§ 371 Date: Apr. 19, 2021

Related U.S. Application Data
(60) Provisional application No. 62/749,785, filed on Oct. 24, 2018.

(51) Int. Cl. G01N 33/94; A61K 49/00; A61K 38/17; A61P 27/04

(52) U.S. Cl. CPC G01N 33/9446; A61K 49/0043; G01N 2800/52; A61P 27/04; G01N 2800/16; A61K 38/1729

(57) ABSTRACT

A kit and methods for diagnosing and treating a dry eye disease or other ocular disease are provided, which include determining whether a reduced level of a histatin is present in a biological fluid sample from a subject.

### INTRODUCTION

This application claims benefit of priority to U.S. Provisional Patent Application Ser. No. 62/749,785, filed Oct. 24, 2018, the content of which is incorporated herein by reference in its entirety.

This invention was made with government support under grant nos. EY024339, EY023656, EY024966 and EY001792 awarded by the National Institutes of Health. The government has certain rights in this invention.

### BACKGROUND

Dry eye disease (DED) is a common disorder that affects millions of patients. Aqueous deficient DED (ADDS) is associated with lacrimal gland dysfunction and loss of the aqueous layer of tears. Several causes for ADDE include, but are not limited to, Sjögren's syndrome (SS) and ocular Graft-versus-Host Disease (oGVHD). Loss of the aqueous layer of the tear film can be associated with ocular surface inflammation, tear hyperosmolarity and a reduction of a number of intrinsic tear components, like lacritin. Characterizing which components of tears are lost in ADDS provides an understanding of the pathophysiology of disease, advances development of diagnostic measures and supports rational therapeutic replacement of diminished components of the tear film.

Histatins are an important class of endogenous anti-microbial peptides (AMP). Other exemplary AMPs include LL-37 and β-defensins. Histatin peptides are a histidine-rich family of 12 peptides (histatin 1-12), arising from two genes HTN1 and HTN3. Histatins were first described as anti-fungal agents in saliva, but have since been found to have anti-viral, anti-bacterial, wound healing and even anti-inflammatory activities. The most common variants of histatin peptides in saliva are H1, H3 and H5, which typically represent 20-30% of the total salivary histatin pool. In whole saliva, the concentration of H1 without stimulation is 12.9±3.7 μg/ml. In saliva, H2, H4, H6-H12 are formed as proteolytic fragments of H1, H3 and H5. Saliva of patients with rheumatoid arthritis with oral sicca symptoms has been noted to have decreased levels of histatins, and there has been interest in testing the use of histatins as markers of disease (Jensen, et al. (1997) Oral Diseases 3:254-261; Winiarczyk, et al. (2018) Graefes Arch. Clin. Exp. Ophthalmol. 256:1127-1139; Giannobile, et al. (2000) Periodontol. 50:52-64).

There has been some interest in the potential role of histatin peptides in the ocular surface and the lacrimal functional unit. In particular, histatins have been shown to be present in the ocular surface epithelia (Huang, et al. (2007) Curr. Eye Res. 32:595-609) and histatin 5 has been detected on Schirmer tear strip samples from healthy subjects (Steele, et al. (2002) Investig. Ophthalmol. Visual Sci. 43:98; Steele, et al. (2006) Clin. Chem. 52(S6):A20-A21). It was also demonstrated that H1 is present in epithelia of accessory lacrimal glands of humans (Ali, et al. (2017) Curr. Eye Res. 42:491-7; Shah, et al. (2016) PLoS One 11(1):e0148018; Ubels, et al. (2012) Investig. Ophthalmol. Visual Sci. 53:6738-6747), and that H1 can promote migration of human corneal epithelia (Shah, et al. (2017) PLoS One 12(5):e0178030). In this respect, U.S. Pat. No. 10,413,587 B2 teaches that histatins may be used for corneal wound healing and as a treatment for ocular surface disease. Similarly, synthetic histatins composed of combinations of functional domains of natural histatins separated by exogenous linkers have been described for the treatment of ocular diseases or conditions (US 2018/0327468 A1).

### SUMMARY OF THE INVENTION

This invention provides a method for diagnosing a dry eye disease by (a) determining in a biological fluid sample from a subject a level of one or more histatin peptides and (b) comparing said level to a reference level, wherein a reduced level of the one or more histatin peptides as compared to the reference level indicates that the subject has a dry eye disease. In some aspects, the dry eye disease is ADDE disease. In certain aspects, the one or more histatins are selected from the group of histatin 1, histatin 2, histatin 3, histatin 4, histatin 5, histatin 6, histatin 7, histatin 8, histatin 9, histatin 10, histatin 11, and histatin 12. In other aspects, the level of the one or more histatin peptides is reduced by at least 2-fold compared to the reference level and/or the level of the one or more histatin peptides is less than 5 μg/ml. While the method may rely on histatin levels alone to diagnose dry eye disease, the method may further include a test to assess the subject's tear film quality (e.g., assessed by measuring tear osmolarity, tear production, tear break-up time, or a combination thereof) or ocular surface health (e.g., assessed by corneal fluorescein staining, Ocular Surface Disease Index scoring, Meiboscale scoring, Dry Eye Questionnaire scoring, or a combination thereof). Upon diagnosis of a dry eye disease, the method may further include the step of administering to the subject having a dry eye disease one or a combination of an anti-inflammatory agent, an immunosuppressive agent, a glucocorticoid, a cytostatic agent, an alkylating agent, an antimetabolic agent, a cytotoxic antibiotic, an opioid or a histatin.

The present invention further provides a method for selecting and treating a subject for a dry eye disease, by (a) determining in a biological fluid sample from a subject a level of one or more histatin peptides; (b) comparing said level to a reference level; and (c) treating the subject with one or a combination of an anti-inflammatory agent, an immunosuppressive agent, a glucocorticoid, a cytostatic agent, an alkylating agent, an antimetabolic agent, a cytotoxic antibiotic, an opioid or a histatin when the level of the one or more histatin peptides are reduced in biological fluid sample from the subject compared to the reference level. In some aspects, the dry eye disease is ADDE disease. In certain aspects, the one or more histatins are selected from the group of histatin 1, histatin 2, histatin 3, histatin 4, histatin 5, histatin 6, histatin 7, histatin 8, histatin 9, histatin 10, histatin 11, and histatin 12. In other aspects, the level of the one or more histatin peptides is reduced by at least 2-fold compared to the reference level and/or the level of the one or more histatin peptides is less than 5 μg/ml.

This invention further provides a method for treating a dry eye disease or other ocular disease by administering to a subject having a dry eye disease or other ocular disease an effective amount of one or more histatins. In certain aspects, the one or more histatins are selected from the group of histatin 1, histatin 2, histatin 3, histatin 4, histatin 5, histatin 6, histatin 7, histatin 8, histatin 9, histatin 10, histatin 11, and histatin 12. In other aspects, the other ocular disease is ocular surface disease, ocular or intraocular inflammation, ocular wounding, ocular surface wounding, ocular epithelial dysfunction or damage, intraocular or ocular surface malignancy.

This invention also provides a kit for diagnosing dry eye disease, which includes (a) a device for collecting tear fluid, and (b) an antibody that selectively binds histatin 1, an antibody that selectively binds histatin 2, an antibody that selectively binds histatin 3, an antibody that selectively binds histatin 4, an antibody that selectively binds histatin 5, an antibody that selectively binds histatin 6, an antibody that selectively binds histatin 7, an antibody that selectively binds histatin 8, an antibody that selectively binds histatin 9, an antibody that selectively binds histatin 10, an antibody that selectively binds histatin 11, an antibody that selectively binds histatin 12, or a combination thereof. While the kit may be used to only determine histatin levels, the kit may further include a test to assess the subject's tear film quality (e.g., assessed by measuring tear osmolarity, tear production, tear break-up time, or a combination thereof) or ocular surface health (e.g., assessed by corneal fluorescein staining, Ocular Surface Disease Index scoring, Meiboscale scoring, Dry Eye Questionnaire scoring, or a combination thereof).

### BRIEF DESCRIPTION OF THE DRAWINGS

FIG. 1 shows ELISA analysis of human tear samples/ocular surface washings of normal, Sjögren's syndrome (SS) and ocular Graft-versus-Host Disease (oGVHD) patients. Histatin 1 (H1) concentration is represented in ng/ml and each measurement (each eye) is represented by a square (normal), diamond (oGVHD) or circle (SS). The mean concentration is noted by the black bar. Statistical comparisons are noted and demonstrated statistically significant p<0.05 differences between normal and all ADDS patients, but no significant (ns) difference between oGVHD and SS.

FIG. 2 shows the ability of H1 concentration to detect patients with high Ocular Surface Disease Index (OSDI) or low Schirmer I values. Patients were reclassified to groups based on their OSDI scores and Schirmer I measurements. Based on OSDI classification, the H1 level was significantly lower in the high OSDI (>13, “diseased”) than the normal group (≤13). Based on Schirmer I classification, H1 level was significantly lower in the low Schirmer I (<10, “diseased”) than the normal group (10).

FIG. 3 shows Multiple Reaction Monitoring (MRM) testing detecting H3 in synthetic peptide spiked solution (positive control), saliva, and tears (two normal, non-ADDE patients).

FIG. 4 shows Multiple Reaction Monitoring (MRM) testing detecting H5 in synthetic peptide spiked solution (positive control), saliva, and tears (two normal, non-ADDS patients).

FIG. 5 shows cell viability testing using tetrazolium salt (WST) assays and comparing HCE viability when exposed to control (Un), toxic preservative (benzalkonium chloride (BAK)), or co-treatment with H1 or H5. Experiments were performed in triplicate. *<0.05, **<0.01. Bar represents SEM.

FIG. 6. shows that treatment with H5 reduces symptoms of dry eye disease in a murine model (BAK exposure) in a statistically significant manner by 7 days (n=5 per condition). Results are based upon the National Eye Institute/Industry (NEI) grading scale (Lemp (1995) CLAO J. 21:221-232) in animals treated with H5 or Balanced Salt Solution (BSS). All experiments were performed in triplicate. *<0.05, **<0.01. Bar represents SEM.

### DETAILED DESCRIPTION OF THE INVENTION

It has now been found that histatins are present in human tears and that the histatin levels, in particular H1, H3 and H5, are reduced in subjects with dry eye disease. Specifically, reduced H1 levels are observed in subjects with Sjögren's syndrome (SS) and ocular Graft-versus-Host Disease (oGVHD) patients as compared to asymptomatic and clinically healthy controls (e.g., subjects that do not have any dry eye symptoms or an eye disorder) and H1 levels have been shown to significantly correlate with conventional clinical indices of aqueous deficiency dry eye (ADDE). Accordingly, the present invention provides methods of diagnosing a dry eye disease or other ocular disease and methods of selecting a subject for treatment of a dry eye disease or other ocular disease. These methods include the steps of determining in a biological fluid sample from a subject the level or amount of one or more histatin peptides and comparing said level to a control or reference level, wherein a reduced histatin peptide level compared to the control or reference level is indicative of dry eye disease or other ocular disease or a subject in need of treatment of the same.

The term “dry eye disease” refers to a multifactorial disease of the tears and ocular surface (including the cornea, conjunctiva, and eye lids), which results in symptoms of discomfort, visual disturbance and tear film instability with potential damage to the ocular surface, as defined by the “The Definition and Classification of Dry Eye Disease: Guidelines from the 2007 International Dry Eye Work Shop” ((2007) Ocul. Surf. 5(2):75-92). Dry eye can be accompanied by increased osmolarity of the tear film and inflammation of the ocular surface. Dry eye disease includes dry eye syndrome, keratoconjunctivitis sicca (KCS), dysfunctional tear syndrome, lacrimal keratoconjunctivitis, evaporative tear deficiency, aqueous deficient dry eye, and LASIK-induced neurotrophic epitheliopathy (LE).

In particular embodiments, the dry eye disease in the methods of this invention is aqueous deficient dry eye or ADDS. ADDE results from reduced lacrimal tear secretion and can be further subdivided to Sjögren's syndrome (the lacrimal and salivary glands are targeted by an autoimmune process, e.g., rheumatoid arthritis), non-Sjögren's syndrome dry eye (lacrimal dysfunction, but the systemic autoimmune features of Sjögren's syndrome are excluded, e.g., age-related dry eye), and ocular Graft-versus-Host Disease (oGVHD). In accordance with this invention, reduced histatin levels are correlated with dry eye disease, preferably ADDS, more preferably ADDS associated with Sjögren's syndrome and oGVHD.

As is known in the art, there are multiple members of the histatin family of peptides, which are the products of two different genes, HTN1 and HTN3. The sequences of the main human histatin family peptide members are provided in Table 1. Preferably, reduced levels of H1, H3 and H5 are correlated with dry eye disease, preferably ADDE associated with Sjögren's syndrome and oGVHD. However, it is posited that the levels of H2, H4, H6, H7, H8, H9, H10, H11 and/or H12 may also be reduced in biological fluids of subjects with a dry eye disease. Accordingly, this invention embraces determining the levels of one or more histatins selected from the group of H1, H2, H3, H4, H5, H6, H7, H8, H9, H10, H11 and/or H12 in accordance with the methods of this invention. In some aspects, the level of H1, H3 and/or H5 peptide is determined. In other aspects, the level of H1 peptide is determined.

TABLE 1

| | | SEQ |
|---|---|---|
| | | ID |
| Histatin | Sequence | NO: |
| H1 | DSpHEKRHHGYRRKFHEKHHSHREFPFYGDYG | 1 |
| | SNYLYDN | |
| H2 | RKFHEKHHSHREFPFYGDYGSNYLYDN | 2 |
| H3 | DSHAKRHHGYKRKFHEKHHSHRGYRSNYLYDN | 3 |
| H4 | KFHEKHHSHRGYRSNYLYDN | 4 |
| H5 | DSHAKRHHGYKRKFHEKHHSHRGY | 5 |
| H6 | DSHAKRHHGYKRKFHEKHHSHRGYR | 6 |
| H7 | RKFHEKHHSHRGY | 7 |
| H8 | KFHEKHHSHRGY | 8 |
| H9 | RKFHEKHHSHRGYR | 9 |
| H10 | KFHEKHHSHRGYR | 10 |
| H11 | KRHHGYKR | 11 |
| H12 | KRHHGYK | 12 |

“Sp” or “S(PO₃)” denotes phosphorylated serine.

For the purposes of this invention, a biological fluid sample is intended to include, but is not limited to, tears, saliva, serum, or urine. Ideally, the biological fluid sample from the subject is a tear fluid sample. In some aspects, the biological fluid sample is a tear fluid sample from a subject suspected of or at risk of having dry eye disease or another ocular disease.

The level or content of a histatin peptide in a biological fluid sample may be determined or measured using any suitable assay for detecting and optionally quantifying a peptide. The level of histatin peptide may be an absolute amount or an amount relative to a control or reference value (e.g., a value or level present in a control (healthy) subject that does not have a dry eye disease or other ocular disease). By the terms “control value,” “control level,” “reference value” or “reference level” is meant a value or level that is used for comparative purposes. In some embodiments, a control or reference value can be a threshold value. In some embodiments, a control or reference value can be a level or value measured in a healthy subject, e.g., a subject that does not present with one or more symptoms of a dry eye disease or a subject that has not been diagnosed as having a dry eye disease or other ocular disease. In some embodiments, the control or reference value is determined in a cohort of reference subjects. In some embodiments, the control or reference value is statistically determined in a cohort of reference subjects, e.g., is the median, mean, or a percentile (e.g., tertile, quartile, quintile) cut-off value (e.g., the top percentile, e.g., top tertile, quartile, or quintile cut-off value) in a cohort of reference subjects.

In some aspects, an amount of one or more histatin peptides in a biological fluid sample is determined using multiple reaction monitoring (MRM) or selected reaction monitoring (SRM) mass spectrometry. In such targeted assays, peptides of interest are selected based on their parent ion mass in first quadrupole (Q1) and fragmented in the second quadrupole (Q2 acts as a collision cell) to generate product ions. Preselected pairs of parent and product ions (a.k.a. transitions) are then monitored by the third quadrupole (Q3). After this two-step filtering mechanism, biological background is mostly removed, leading to the detection of preselected peptides with high specificity and sensitivity. A modern triple quadrupole mass spectrometer is capable of scanning hundreds of transitions in a single experiment to detect multiple peptides simultaneously. In addition, if stable isotope-labeled peptides with known quantity are spiked into a sample as internal standards, the absolute quantity of these preselected peptides can be determined with high accuracy.

Alternatively, the level or content of one or more histatins in a biological fluid sample may be determined using antibodies that selectively binds the one or more histatin peptides. An antibody that is selective for the particular histatin peptide may be polyclonal or monoclonal, and is not limited by the animal species from which the antibody is derived. An antibody that selectively binds a histatin peptide ideally has a binding affinity (e.g., expressed as binding affinity dissociation constant or K_{D}) for a particular histatin that is at least 2-, 5-, 10-, 20-, 50- or 100-fold higher than the binding affinity for other histatins. The antibody may be a full-length immunoglobulin or antibody fragment having antigen-binding activity. Examples of antibody fragments include, but are not limited to, a Fab fragment and a F(ab′) fragment. Antibody and antibody fragments of use in quantifying histatin peptide levels are known in the art and available from commercial sources such as Abcam (Cambridge, Mass.), LifeSpan BioSciences (Seattle, Wash.), Antibodies-online (Atlanta, Ga.), Novus Biologicals (Littleton, Colo.), MyBioSource.com (San Diego, Calif.), United States Biological (Salem, Mass.), and Creative Biolabs (Shirley, N.Y.).

When the level of the histatin peptide is measured in a biological fluid sample, the histatin peptide may be detected using, for example, an immunohistochemical technique or western blot analysis and the content of the histatin peptide thus detected in the sample can be calculated from a predetermined standard curve. Alternatively, the level of the histatin peptide can be determined using a quantitative method such as ELISA (enzyme-linked immunosorbent assay) including direct competitive ELISA, indirect competitive ELISA, and sandwich ELISA, RIA (radioimmunoassay), flowmetry, or immunochromatography.

When an antibody or antibody fragment is used in determining the level of a histatin peptide, preferably the antibody that is selective for the histatin (i.e., the primary antibody), or a secondary antibody that binds thereto, is labeled for the purpose of visualization. Examples of the labeling substance include, but not limited to, fluorescent substances (e.g., FITC, rhodamine, and phalloidine), colloidal particles such as gold, fluorescent microbeads (Luminex Corporation), heavy metals (e.g., gold and platinum), chromoproteins (e.g., phycoerythrin and phycocyanin), radioisotopes (e.g., ¹H, ¹⁴C, ³²P, ³⁵S, ¹²⁵I and ¹³¹I), enzymes (e.g., peroxidase and alkaline phosphatase), biotin and streptavidin.

In some aspects, a subject is diagnosed with a dry eye disease or other ocular disease when the level or content of histatin in the subject's biological fluid sample is at least 2-fold, or preferably at least 5-fold, or more preferably at least 10-fold lower than the control or reference level. In certain aspects, a subject is diagnosed with ADDE when the level or content of histatin in the subject's tear fluid is at least 2-fold, or preferably at least 5-fold, or more preferably at least 10-fold lower than the control or reference level. In particular aspects, a subject is diagnosed with ADDS when the level or content of H1, H3 and/or H5 in the subject's tear fluid is at least 2-fold, or preferably at least 5-fold, or more preferably at least 10-fold lower than the control or reference level.

In some aspects, a subject is diagnosed with a dry eye disease when the level or content of at least one histatin in the subject's biological fluid sample is less than 5 μg/ml, or preferably less than 1 μg/ml, or more preferably less than 600 ng/ml, or most preferably less than 400 ng/ml. In certain aspects, a subject is determined to have ADDE when the level or content of at least one histatin in the subject's tear fluid is less than 5 μg/ml, or preferably less than 1 μg/ml, or more preferably less than 600 ng/ml, or most preferably less than 400 ng/ml. In particular aspects, a subject is determined to have ADDE when the level or content of H1, H3 and/or H5 in the subject's tear fluid is less than 5 μg/ml, or preferably less than 1 μg/ml, or more preferably less than 600 ng/ml, or most preferably less than 400 ng/ml lower than the control or reference level.

In some aspects, the methods of this invention further include determining, or alternatively obtaining, providing, or using previously determined information regarding one or more additional symptoms of dry eye disease or other ocular disease in the subject. Such symptoms include, but are not limited to, pain or a burning sensation in the eyes, tear evaporation, hyperosmolarity, instability of the tear film, bacterial growth on the lid margin, and/or presence of ocular surface inflammation and/or damage. In this respect, some aspects of the methods of the invention further include assessing one or more (e.g., two, three, four, or five) additional symptoms of dry eye disease in the subject including one or more of dry eyes, pain or a burning sensation in the eyes, hyperosmolarity, instability of the tear film, increased bacterial growth on the lid margin, and ocular surface inflammation and damage. In certain aspects, the methods of the invention further include performing a test to determine tear quality (e.g., a test to determine tear osmolarity, tear production or tear break-up time) or a test to assess ocular surface health (e.g., corneal fluorescein staining, Ocular Surface Disease Index (OSDI) scoring, Meiboscale scoring, and Dry Eye Questionnaire (DEQ) scoring) and correlating the same with histatin levels. Methods for determining tear osmolarity, tear production and tear break-up time, and performing corneal fluorescein staining, OSDI scoring, and DEQ scoring are known in the art. In some embodiments, one or more of a decrease in tear production or an elevation in corneal fluorescein staining, tear osmolarity, tear break-up time, Meiboscale score, or OSDI score further indicates that the subject has a dry eye disease.

By the term “corneal fluorescein staining” is meant a clinical procedure that is used to assess corneal injury, corneal defect(s), or defects in the tear film in a subject. In some embodiments of this procedure, a piece of hydrated blotting paper containing a dye (e.g., fluorescein) is brought into contact with the subject's eye, who is then asked to blink, and the subject's eye is visualized with a blue light. Any corneal abrasions, corneal defect(s), or structural inconsistencies in the tear film will be observed by a speckled, uneven distribution of the dye in the eye of the subject.

“Tear osmolarity” refers to the concentration of a solution expressed as the total number of solute particles per liter. There are several diagnostic technologies that can be used for tear film osmolarity testing. The freezing point depression method of requires only a 0.2 μL sample of tears and is highly accurate. According to this method, the sample is supercooled to its freezing point and osmolarity is calculated by comparing the sample's freezing point measurement to known standards. A second approach, vapor pressure osmometry, is a relatively simple alternative method to measure tear osmolarity. For this method, a sample is inserted into the instrument, where it is diffused onto a paper disk inside a small, enclosed chamber. As the sample evaporates, a sensor (a thermocouple hygrometer) measures the dew point temperature within the chamber. The difference between the dew point temperature and the baseline temperature is the dew point temperature depression. Calculating this difference provides the vapor pressure of the solution, i.e., osmolarity. As a further alternative, an electrical impedance osmometer is used to measure osmolarity.

The “tear production test” or the “Schirmer's Test” refers to a test that measures the volume of tears produced, and is performed by placing a small strip of filter paper inside the lower eyelid (conjunctival sac) of each eye for several minutes, allowing tear fluid to be drawn into the filter paper by capillary action. The paper is then removed and the amount of moisture is measured in millimeters. Typically, a measurement of less than 5 mm indicates dry eye.

By the term “tear break-up time” is meant a clinical score that indicates the stability of the tear film in a subject. A number of clinical tests are available for determining the tear break-up time in a subject. In some embodiments, the tear break-up time is assessed by adding a sodium fluorescein dye to a subject's eye; observing the dye, while the patient avoids blinking; and recording the elapsed amount of time before tiny dry spots appear on the cornea. In such embodiments, the longer it takes for the dry spots to develop, the more stable the tear film is in the subject. A variety of additional assays for determining the tear break-up time in a subject are available in the art.

A “dry eye questionnaire” refers to a set of questions, the answers of which indicate whether a subject has a dry eye disease. Examples of dry eye questionnaires include, e.g., 0-10 Cooling Scale, the 4-Symptom Questionnaire, the Ocular Surface Disease Index (OSDI, Allergan), the Symptom Assessment iN Dry Eye (SANDE), the Standard Patient Evaluation of Eye Dryness (SPEED, TearScience), the Dry Eye Questionnaire (DEQ, TearLab), the McMonnies Questionnaire, the Subjective Evaluation of Symptom of Dryness (SESoD, Allergan), the Impact of Dry Eye on Everyday Life (IDEEL, Alcon) and the Dry Eye-Related Quality-of-Life Score Questionnaire (DEQS, Dry Eye Society), or a combination thereof. In some embodiments, the dry eye questionnaire is OSDI.

“OSDI” refers to a set of 12 questions answered by a subject suspected of having a dry eye disease. The questions include: (I) Have you experienced any of the following during the last week: Eyes that are sensitive to light? Eyes that feel gritty? Painful or sore eyes? Blurred vision? Poor vision? (II) Have problems with your eyes limited you in performing any of the following during the last week: Reading? Driving at night? Working with a computer or bank machine (ATM)? Watching TV? (III) Have your eyes felt uncomfortable in any of the following situations during the last week: Windy conditions? Places or areas with low humidity (very dry)? Areas that are air conditioned?) by circling the number that best represented each answer: 4 (all of the time), 3 (most of the time), 2 (half of the time), 1 (some of the time) or 0 (none of the time). To obtain the total score for the questionnaire, the final score is calculated using the following formula: (A) Add subtotals from Sections (I), (II), and (III); (B) Determine total number of questions answered from Sections (I), (II), and (III)(do not include N/A); (C) Final OSDI score=A×25 divided by B.

In some aspects, a subject is diagnosed with a dry eye disease, in particular ADDE, when the level or content one or more histatins (e.g., H1, H3 and/or H5) in the subject's tear fluid is at least 2-fold, at least 5-fold, or at least 10-fold lower than the control or reference level and the subject has an OSDI score of at least 13 and/or a Schirmer I measurement of less than 10. In other aspects, a subject is diagnosed with a dry eye disease, in particular ADDE, when the level or content one or more histatins (e.g., H1, H3 and/or H5) in the subject's tear fluid is less than 5 μg/ml, less than 1 μg/ml, less than 600 ng/ml, or less than 400 ng/ml and the subject has an OSDI score of at least 13 and/or a Schirmer I measurement of less than 10.

In some embodiments, the methods can be performed by a health care professional (e.g., a physician, a physician's assistant, a nurse, a nurse's assistant, and a laboratory technician). In some embodiments, the subject can be a child, a teenager, or an adult (e.g., at least 18, 20, 25, 30, 35, 40, 45, 50, 55, 60, 65, 70, 75, 80, 85, or years old). A subject can, e.g., present with one or more (e.g., at least two, three, or four) of the symptoms of dry eye disease described herein. In some embodiments, a subject, e.g., may not present with a symptom of dry eye disease that can be easily detected by basic examination of an eye(s) of the subject.

The methods described herein can be periodically performed (e.g., at least once a month, once every six months, or once a year) on a subject that has an increased risk of developing a dry eye disease or other ocular disease (e.g., woman in menopause or a subject having androgen deficiency, Sjogren's syndrome, psoriasis, rosacea, hypertension, or benign prostatic hyperplasia, or a subject that is taking or a subject that was previously administered one or more of an antiandrogen, a postmenopausal hormone therapy (e.g., estrogens and progestins), an antihistamine, an antidepressant, and a retinoid)). Some embodiments further include recording the results of the test in the subject's medical records (e.g., recording the results in a computer readable medium).

To facilitate diagnosis, the present invention also provides a kit for determining whether a subject has a dry eye disease or other ocular disease. The kit of the invention includes a device for collecting a biological fluid sample and one or more agents for determining the level of one or more histatins. Ideally, the agents for determining histatin levels are one or more anti-histatin antibodies, i.e., an antibody that selectively binds H1, H2, H3, H4, H5, H6, H7, H8, H9, H10, H11 or H12. Preferably, the kit includes an antibody that selectively binds to H1, an antibody that selectively binds to H3 and/or an antibody that selectively binds to H5. The device for collecting biological fluid sample can be, e.g., a glass capillary tube, a test strip (e.g., a hydrogel strip), or contact lens. A tear collecting device is preferably a test strip or a contact lens.

In certain aspects, the kit includes a device for determining the level of one or more histatins, wherein the device includes (a) a test strip configured to receive a biological fluid sample, e.g., a tear or saliva sample from a patient; and (b) a reagent pad containing one or more anti-histatin antibodies, that, upon contact with the biological fluid sample, undergo a reaction configured to produce a detectable signal, wherein the intensity of the signal is proportional to the amount of histatin in the biological fluid sample, and wherein the test strip is configured to deliver the biological fluid sample to the reagent pad. A detectable signal, for example, can be electrical signals (electrochemical assays), or optical signals (enzyme assays, immunoassays or competitive binding assays). Optical signals refers to changes in the optical properties, including, but not limited to, a color formation, a change in color, fluorescence, luminescence, chemiluminescence, changes in fluorescence or luminescence intensity, changes in fluorescence or luminescence lifetimes, fluorescent anisotropy or polarization, a spectral shift of the emission spectrum, time-resolved anisotropy decay, and the like.

In some aspects, the kit further includes a test to assess the subject's tear film quality (e.g., tear osmolarity, tear production, or tear break-up time) or ocular surface health (e.g., a corneal fluorescein stain, Ocular Surface Disease Index test, Meiboscale test, or Dry Eye Questionnaire test).

Once a subject has been identified as having a dry eye disease or other ocular disease based upon the level of one or more histatins and optionally one or more additional symptoms, the subject can be treated with one or a combination of agents of use in treating dry eye disease and/or an underlying etiology associated with an ocular disease. Such treatments can include, e.g., the administration of one or a combination of an anti-inflammatory agent (e.g., a TNF binding protein, interferon beta, interferon gamma, or anti-inflammatory antibiotic such as azithromycin, doxycycline, clarithromycin, dirithromycin, erythromycin, roxithromycin, telithromycin, carbomycin A, josamycin, kitasamycin, midecamycin, oleandomycin, solithomycin, spiramycin, troleandomycin, or tylocine), an immunosuppressive agent (cyclosporin A or analogs thereof, Tacrolimus/FK506, Sirolimus/Rapamycin, mycophenolate, an anti-thymocyte globulin sold under the tradenames ATGAM® or THYMOGLOBULIN®, an anti-CD3 antibody OKT3, any antibody against the T-cell receptor, or any antibody against IL-2, e.g., basiliximab or declizumab), a glucocorticoid, a cytostatic agent, an alkylating agent (e.g., nitrogen mustards/cyclophosphamide, nitrosoureas, platinum compounds), an antimetabolic agent (e.g., methotrexate, any folic acid analog, azathioprine, mercaptopurine, any purine analog, any pyrimidine analog, any inhibitor of protein synthesis), a cytotoxic antibiotic (e.g., dactinomycin, an anthracycline, mitomycin C, bleomycin, mithramycin), or an opioid.

Moreover, the invention provides for the administration of a histatin or variant thereof, e.g., H1, H2, H3, H4, H5, H6, H7, H8, H9, H10, H11 and/or H12, in the treatment of a dry eye disease (e.g., ADDE) or other ocular disease, either alone or in combination with one or more of the above-referenced therapeutics. Other ocular diseases that may be treated in accordance with this aspect include, but are not limited to, ocular surface disease, ocular or intraocular inflammation, ocular wounding, ocular surface wounding, ocular epithelial dysfunction or damage, intraocular or ocular surface malignancy. In accordance with this aspect, the histatin is provided in an ophthalmic formulation including the histatin in combination with an acceptable carrier. The histatin peptide may be produced by conventional methods including synthetic peptide synthesis or recombinant production and may be based upon the sequences provided herein in Table 1, or analogs or derivatives thereof. In certain embodiments, the histatin used in this aspect of the invention is H1, H3 and/or H5.

Acceptable formulation materials preferably are nontoxic to recipients at the dosages and concentrations employed. The formulation may contain materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as PLURONICS, PEG, sorbitan esters, polysorbates such as polysorbate 20 and polysorbate 80, TRITON, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, Remington's Pharmaceutical Sciences, Id.

The primary carrier or excipient in a pharmaceutical composition may be either aqueous or nonaqueous in nature. For example, a suitable carrier or excipient may be water for injection, physiological saline solution or artificial cerebrospinal fluid, possibly supplemented with other materials common in compositions for parenteral administration. Neutral buffered saline or saline mixed with serum albumin are further exemplary excipients. Pharmaceutical compositions can include Tris buffer of about pH 7.0-8.5, or acetate buffer of about pH 4.0-5.5, which may further include sorbitol or a suitable substitute. Compositions of the invention may be prepared for storage by mixing the selected composition having the desired degree of purity with optional formulation agents (Remington's Pharmaceutical Sciences, Id.) in the form of a lyophilized cake or an aqueous solution. Further, the histatins of the invention may be formulated as a lyophilizate using appropriate excipients such as sucrose.

The histatin may be administered in any form suitable for ocular drug administration, e.g., as a solution, suspension, ointment, gel, liposomal dispersion, colloidal microparticle suspension, or the like, or in an ocular insert, e.g., in an optionally biodegradable controlled release polymeric matrix. Preferably, the ophthalmic formulation contains an effective amount of the histatin to improve or restore the level of the histatin in the tear fluid, e.g., to near normal levels.

The following non-limiting examples are provided to further illustrate the present invention.

### Example 1: Materials and Methods

Subject Participants and Classification. This research study was conducted under a University of Illinois at Chicago Institutional Review Board approved protocol, adhering to the tenets of the Declaration of Helsinki and the Health Insurance and Portability and Accountability Act. Symptomatic patients with dry eye disease (DED) and asymptomatic healthy control subjects were enrolled, and informed consent was obtained from all participants after the nature and possible consequences of the research were explained.

Participants were divided into the following two groups: (i) normal controls (n=11, 22 eyes) (2) ADDS (n=11, 22 eyes), which included two subgroups: (a) Sjögren's syndrome (SS) group (n=4, 8 eyes) and (b) ocular Graft-versus-Host Disease (oGVHD) (n=7, 14 eyes). This sample size allowed detection of a group mean difference of 1.26 standard deviation for a power of 0.8 and a significance level of 0.05.

Classification of patients was determined as follows. The control group included individuals with no ocular symptoms, a Schirmer I test without anesthesia result exceeding 10 mm at 5 minutes and an Ocular Surface Disease Index (OSDI) score below 13. Patients were considered to have ADDE if their Schirmer I at 5 minutes was less than 10 mm of wetting, vital dye staining of the cornea ≥1 on the NEI-CSS, and an OSDI>13 (Bron, et al. (2007) Ocular Surface 5:108-152; Lemp (1995) CLAO J. 21:221-232). Schirmer I testing is a standard test used as an element in the diagnosis of ADDS (Bron (2001) Surv. Ophthalmol. 45:S221-S226; Wolffsohn, et al. (2017) Ocular Surface 15:539-574). SS diagnosis was based on a preexisting diagnosis from a board-certified rheumatologist with either presence of specific serum auto-antibodies or the presence of a disease-consistent biopsy and concomitant ADDE as noted above. oGVHD diagnosis was based on previously reported consensus guideline criteria (Ogawa, et al. (2013) Sci. Reports 3:3419). Briefly, it included four subjective and four objective criteria, corneal staining, OSDI, Schirmer I, tear production measurements, and conjunctival injection. All patients in the oGVHD group that were included in the study had a diagnosis of “definite” oGVHD.

Examination and testing order during clinical visits were performed in the following order: OSDI scoring, Schirmer I, slit lamp biomicroscopic examination of the ocular surface (using a broad beam at medium intensity), ocular surface washings/tear collection, Vital dye staining and evaluation of the ocular surface with fluorescein.

Schirmer I (Without Anesthesia) Testing and Ocular Surface Disease Index (OSDI). Schirmer I testing was undertaken to measure tear production using Whatman filter strips #41 (Haag-Streit, Essex, UK). After 5 minutes, the Schirmer strip was removed and the length of wetting (mm) was measured. OSDI scores range from 0-100 with higher scores associated with more severe DED or disability. OSDI score was calculated using the following formula, OSDI=[Σ(scores of all the questions)×25]/(total number of questions)(Tibrewal, et al. (2013) Invest. Ophthalmol. Visual Sci. 54:8051-8061; Schiffman, et al. (2000) Arch. Ophthalmol. 118:615-621).

Corneal Staining. Corneal staining score as measured by Lissamine Green dye staining (5 μL of 1% solution applied to each eye) using National Eye Institute-Corneal Staining score (NEI-CSS), grading scale (Lemp (1995) CLAO J. 21:221-232; Hamrah, et al. (2011) Eye (London, England) 25:1429-1434). The NEI scale relies on a chart that divides the cornea into five sections and assigns a value from 0 (absent) to 3 (severe) to each section, based on the density of punctate keratitis, for a maximum of 15 points.

Non-Invasive Tear Breakup Time (NITBUT). NITBUT is the time (in seconds) it takes for distortions to appear in the image of concentric Placido rings that are reflected on the patient's cornea by a keratograph (Oculus, Inc., Arlington, Wash.). Two types of NITBUT are measured by the Keratograph 5M: (i) NITBUT-first is the time at which the first distortion of Placido rings occurs; and (ii) average NITBUT is the average time of first breakup incidents in different locations in a corneal diameter of 8 mm. The average NITBUT is provided in Table 3 to compare the tear breakup time amongst ADDE subgroups (oGVHD and SS).

Meibomian Gland Analysis. Meibomian Gland imaging was performed using LipiView II Ocular Surface Interferometer (TearScience, Morrisville, N.C.). Meibomian gland dropout was graded using a 0 to 4 scale based on the area of Meibomian gland loss (0, 0%; 1, <25%; 2, 25%-50%; 3, 51%-75%; and 4, >75%). The score was recorded as “Meiboscale” for each eye (Pult & Riede-Pult (2013) Contact Lens Anterior Eye 36:22-27; Yeotikar, et al. (2016) Invest. Ophthalmol. Visual Sci. 57:3996-4007).

Tear Collection. Ocular surface washings (OSW) were performed for tear collection, following published protocols (Tibrewal, et al. (2013) Invest. Ophthalmol. Visual Sci. 54:8051-8061; Caffery, et al. (2008) Optomet. Vision Sci. 85:661-667). OSW were preferred, as opposed to direct capture of tears with microcapillary tube, as sufficient tear volume was not present to directly collect tears from patients with ADDE. In order to maintain comparability, the same method of collection for tears was performed for normal patients. Briefly, ocular surface washings were obtained using a 50 μl drop of preservative-free artificial tears sold under the tradename REFRESH OPTIVE® Sensitive (Allergan, Inc.) instilled in the eye by pipette, and OSW were performed then after a 2-minute period, wherein tears from the lower lid margin and inferior fornix of all patients were collected using a blunt glass microcapillary tube (5 μL Drummond MICROCAPS®; Thermo Fisher Scientific, Waltham, Mass.). OSW tears were stored in a sterile tube (Thermo Fisher) at −80° C. (Tibrewal, et al. (2013) Invest. Ophthalmol. Visual Sci. 54:8051-8061).

Saliva Collection. Unstimulated whole saliva was collected from normal subjects without known oral diseases including severe periodontitis or dry mouth. Saliva was collected similarly to published protocols (Agha-Hosseini, et al. (2011) Aust. Dent. J. 56:171-174; Takehara, et al. (2013) PLoS One 8:e69059). Briefly, without any stimulation, saliva was allowed to accumulate in the mouth and expelled into a sterile culture dish. Approximately, 2 ml of whole saliva was collected and centrifuged at 14000 RPM at 4° C. for 10 minutes; the supernatant was then used for experimentation. Saliva was collected only for use as a positive biological control to determine the presence or absence of H1 in tears.

Histatin-1 Synthesis. Histatin-1 (H1) peptide was synthesized according to previous published protocol (Shah, et al. (2017) PLoS One 12:e0178030). The sequence of the peptide was, DS(PO₃)HEKRHHGYRRKFHEKHHSHREFPFYGDYGSNYLYDN (SEQ ID NO:1) with phosphorylation of ‘S (Serine)’ and without any other modification. The theoretical size of H1 is 4928.17. Briefly, standard solid phase using Fmoc chemistry was used for the H1 synthesis. The peptide was purified using HPLC and characterized by electrospray ionization mass spectrometry. Lyophilized peptide powder was dissolved in phosphate buffered saline (PBS) for use in all experiments. H1 peptide was synthesized as Trifluoroacetic acid (TFA) salts with >95% purity.

Western Blot, Immuno-dot Blot and Coomassie Staining. Western blot analysis was performed following standard methods (Sarkar, et al. (2012) Invest. Ophthalmol. Visual Sci. 53:1792-1802). Briefly, tear samples (2.5 μl) were electrophoresed on 12% NuPage™ bis-Tris gels (Invitrogen, Carlsbad, Calif.) and transferred to nitrocellulose membranes. Membranes were blocked with Tris-buffered saline containing 3% nonfat dry milk and 0.02% polyethylene glycol sorbitan monolaurate sold under the tradename TWEEN® 20 and incubated with 1:1000 rabbit anti-human Histatin-1 (H1) antibody (Mybiosource, San Diego, Calif.) overnight at 4° C. and with 1:2000 goat anti-rabbit-HRP (BD Biosciences, San Jose, Calif.) as the secondary antibodies for hour. The membranes were developed using X-ray film and ECL Pro solution (PerkinElmer, Waltham, Mass.). Western blot protein quantification was performed using BioDrop (Biochrom Ltd., Cambridge, UK).

For immunodot blot analysis, 4 μl of serial dilutions of synthetic H1 (Syn.H1) were spotted onto a nitrocellulose membrane and dried. Syn.H1 peptide and saliva were used as positive controls and a scrambled peptide and PBS were used as a negative control. Rabbit anti-human H1 antibody or Mouse anti-human H1 antibody (Abcam, Cambridge Mass.) were used for primary antibodies. For detection, Goat anti-Rabbit (conjugated to a fluorescent dye sold under the tradename IRDYE® 680 RD) or Goat anti-mouse (conjugated to a fluorescent dye sold under the tradename IRDYE® 800CW) were used as secondary antibodies. The relative intensity of each band was determined with an imaging system sold under the tradename ODYSSEY® (Li-Cor Biosciences).

For qualitative comparison of the level of total protein between tear samples, gels were stained with Coomassie Brilliant Blue R-250 staining solution (Bio-Rad Hercules, Calif., USA) as has been performed for qualitative comparison of protein levels in tears (Grus, et al. (2001) Electrophoresis 22:1845-1850).

Multiple Reaction Monitoring (MRM). MRM was performed for identification of presence of H1 in tears on an LC-MS/MS system (Agilent 1200 HPLC (Agilent Technologies, Santa Clara, Calif.) coupled with mass spectrometry (QTRAP® 6500; Sciex, Framingham, Mass.)(Seki, et al. (2010) J. Immunol. 184:836). Chromatography was performed using a binary solvent system composed of A: 0.1% formic acid and 5% acetonitrile (ACN) and B: 0.1% formic acid and 95% ACN at a flow rate of 200 μL/minute and mobile phase and reversed-phase columns SB-C18 (2.1 mm×50 mm, particle size 1.8 μm, Agilent Technologies). A gradient was run from 5% B to 60% B over 10 minutes. A standard curve for H1 was constructed using a range of concentrations with Syn.H1. Samples were centrifuged at 14,000 RPM for 9 minutes followed by injection of 10 μL of the supernatant into the LC-MS/MS system. The MRM was operated in the positive ion mode monitoring the 704.9-931.0 m/z, transitions for H1.

Enzyme-Linked Immunosorbent Assay (ELISA). H1 concentration was determined by ELISA detection using a commercially available H1 ELISA kit (MyBioSource, Inc., San Diego, Calif.) following the manufacturer's instructions. Samples were read with a microplate reader (Biotek Synergy H1, Winooski Wis.).

Statistical Analysis. The data were summarized by means and standard errors for continuous variables or by proportions for categorical variables. Demographic characteristics (age, sex) were compared between normal and ADDE groups using t-tests or Chi-square tests. The values of Schirmer I, OSDI and H1 concentration from two eyes were averaged first for each participant then were compared between groups using t-tests. The association between H1 concentration and other measures (age, sex, Schirmer I, OSDI and disease status) were assessed using mixed linear models, which could account for between eye correlation. Finally, logistic regression was used to estimate odds ratios of disease from the averaged H1 levels from two eyes for each individual, followed by Receiver-operator-characteristic (ROC) analysis.

### Example 2: Presence of Histatin-1 in Human Tears and Association with Aqueous Deficient Dry Eye (ADDE)

Patient Demographics and Phenotypic Characteristics. Patient demographic and clinical data are shown in Table 2.

TABLE 2

| Factor | Normal (n = 11) | ADDE (n = 11) | p-value |
|---|---|---|---|
| Age (years) | 44.6 ± 2.6 | 53.5 ± 2.9 | 0.032 |
| Male Sex | 7 (63.6%) | 6 (54.6%) | 0.665 |
| Schirmer I (mm) | 18.3 ± 2.5 | 1.9 ± 0.8 | <0.001 |
| OSDI | 1.9 ± 1.0 | 33.8 ± 6.6 | <0.001 |
| H1 (ng/ml) | 891.6 ± 196.5 | 85.9 ± 63.7 | <0.001 |

Data are presented as mean ± standard error of the mean or %. Schirmer I = Testing, in 5 minutes. OSDI = Ocular Surface Disease Index. H1 = Histatin-1.

Compared to the normal group, the ADDS group had a higher age (53.5±2.9 years vs. 44.6±2.6 years, p=0.032 from t-testing) and a comparable percentage of males (54.6% vs. 63.6%, p=0.665 from Chi-square testing). Consistent with the clinical definition of ADDS, it was observed that the ADDE group had significantly lower Schirmer I measurements (1.9±0.8 mm vs. 18.3±2.5 mm, p<0.001 from t-testing) and higher OSDI scores (33.8±6.6 vs. 1.9±1.0, p<0.001 from t-testing) than the normal group.

With an average Schirmer I of 1.9 mm, the ADDE patient population included severe disease phenotypes. Additional phenotypic data including non-invasive tear break up time (NITBUT), NEI-CSS and Meiboscale scores are provided in Table 3.

TABLE 3

| Factor | SS | oGVHD | p-value |
|---|---|---|---|
| Schirmer I (mm) | 3.2 ± 2.1 | 1.1 ± 0.0 | 0.231 |
| OSDI (0-100) | 48.1 ± 12.3 | 25.6 ± 6.2 | 0.099 |
| Corneal Staining Score (0-15) | 2.4 ± 1.0 | 4.8 ± 1.6 | 0.3045 |
| Average NITBUT (secs) | 4.1 ± 1.3 | 5.5 ± 0.8 | 0.392 |
| Meiboscale score (0-4) | 0.4 ± 0.2 | 1.1 ± 0.1 | 0.0202 |
| H1 (ng/ml) | 197.6 ± 170.2 | 22.0 ± 22.0 | 0.199 |

Data are presented as mean ± standard error of the mean. SS, Sjögren's syndrome. oGVHD, ocular Graft-versus-Host Disease. OSDI, Ocular Surface Disease Index. Schirmer I (mm), Schirmer I (without anesthesia). NIBUT, Non-invasive tear break-up time. H1 = Histatin-1.

Notably, NITBUT (p=0.392) and Corneal Staining Scores (p=0.3045) were not significantly different between ADDE subgroups (SS and oGVHD) whereas, Meiboscale score means were significantly different between SS and ADDE subgroups (SS, 0.4±0.2 and oGVHD 1.1±0.1, p=0.0202).

Histatins are Present in Ocular Surface Washings. Antibody specificity was confirmed using Immune Dot Blot (IDB) over a series of concentrations of synthetic H1. In accordance with this analysis, synthetic H1 peptide was detected at a greater dot density with increasing concentrations of peptide in solution. Scrambled peptide negative control was found to be undetectable. The presence of H1 in normal human tears was then assessed using IDB and western blot analyses. This analysis indicated that H1 peptide was present in ocular surface washings. Positive control (Synthetic H1 (Syn.H1)) and biological positive control (Saliva) showed similar results.

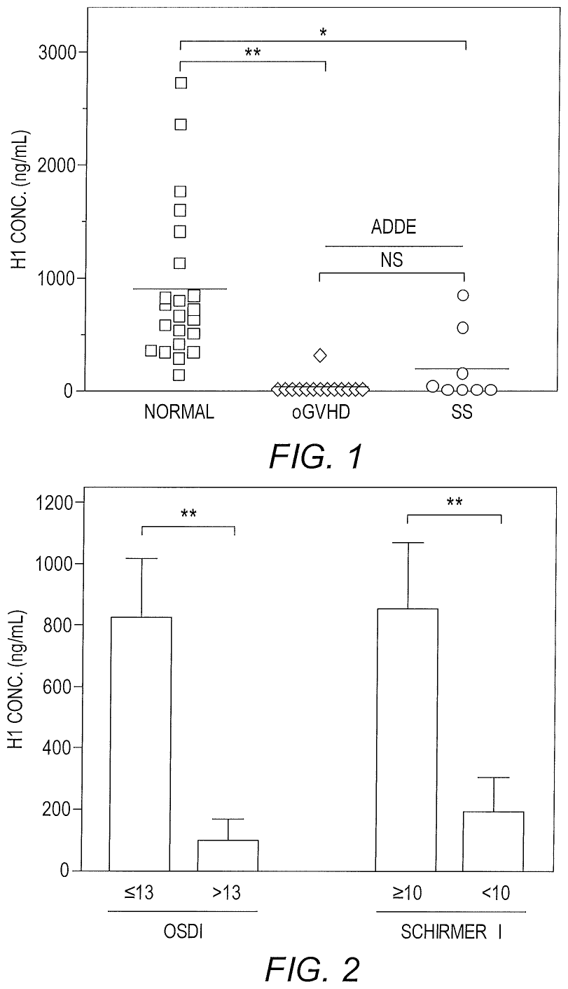

Multiple reaction monitoring (MRM) was then used to identify the presence or absence of H1 in ocular surface washings and to confirm the western blot and IDB results, as this technique is not dependent upon antibody specificity. Targeted MRM detected the presence of H1 in solubilized Syn.H1, normal human tear ocular surface washings and saliva (FIG. 1).

Histatin Levels are Lower in ADDE Patients Than Normal Controls. Ocular surface washings from ADDS patients and normal controls were tested using ELISA (FIG. 1). Random intercept mixed linear modeling with eyes nested in patients indicated that the diseased group had lower H1 concentrations than the normal group (85.9±63.7 ng/ml vs. 891.6±196.5 ng/ml, β (se)=−805.7(206.5), p<0.001). H1 levels were significantly associated with age (β(se)=−36.1(11.5), p=0.002), OSDI score (β(se)=−13.4(5.5), p=0.016), and Schirmer (β(se)=20.9(10.00), p=0.036) (Table 4). By comparison, H1 level did not depend on sex (β(se)=−255.2(272.8), p=0.349).

TABLE 4

| Risk Factor | Odds Ratio | Standard Error | p-value |
|---|---|---|---|
| H1 (100 ng/ml | 0.5 | 0.1 | 0.016 |
| increase)* | | | |
| Age (1 year increase) | 1.1 | 0.1 | 0.055 |
| Male sex | 0.7 | 0.6 | 0.665 |

*H1 = Histatin-1, the mean value between two eyes for each subject was used for odds ratio calculation.
OSDI and Schirmer I testing were not included in this analysis as they were used for disease status classification. The dependent variable is disease status (ADDE vs normal) and the independent variable is H1 with age as the control variable.

To address the association with age, mixed linear modeling was performed to control for age and diagnosis of ADDE. This analysis indicated that H1 association with diagnosis was still significant (β(se)=−612.0(217.4), p=0.005). These results indicate that the difference in H1 concentrations between the diseased and normal subjects could not be accounted for by the differences in age.

In order to determine whether estimates of total protein levels varied with, or were independent of, H1 levels, outliers (high and low H1 concentration of samples with available residual tears for testing in each group: normal, oGVHD, SS) were selected and evaluated by performing gel electrophoresis and Coomassie staining. The results of these tests showed, in general, that there was no strong relationship between total protein levels and H1 concentration, indicating they may vary independently of one another.

Receiver-operator-characteristic (ROC) analysis was then performed, which indicated that using ELISA H1 to classify ADDE status yielded an area under of curve of 0.96 (95% CI=0.87-1.00). By using a cutoff with ELISA H1 of 0.378 ng/ml as a criterion for ADDE, a sensitivity of 90.9% and specificity of 90.9% for ADDE diagnosis was obtained. These results indicate that H1 levels are of diagnostic use for ADDE.

Given the findings that ADDE diagnosis strongly associated with H1 levels, it was determined whether H1 levels were associated with specific diagnostic elements. The association between H1 concentration and disease was then assessed by reclassification of subjects into DED defining criteria: OSDI and Schirmer I. Based on OSDI classification, the H1 level was significantly lower in the diseased group than the normal OSDI group (t(20)=3.2, p=0.004) (FIG. 2). Similarly, based on Schirmer I classification, H1 level was higher in the normal group than the normal Schirmer I group (t(20)=2.9, p=0.010)(FIG. 2). These data therefore indicated that H1 concentration is a useful diagnostic test, which correlates well with gold standard diagnostic measures including OSDI and Schirmer.

### Example 3: Association of Histatin 3 (H3) and Histatin 5 (H5) with ADDE

Similar to the analysis conducted with H1, ocular surface washings from ADDE patients and normal controls were tested for the levels of H5 using ELISA. As demonstrated by the levels presented in Table 5, H5 peptide was diminished in subjects with ADDE (i.e., SS and oGVHD) compared to normal controls.

TABLE 5

| Subject | H5 (ng/mL) | |
|---|---|---|
| Normal | 5.37 | (n = 4) |
| oGVHD | 0.07 | (n = 2) |
| SS | −0.59 | (n = 1) |

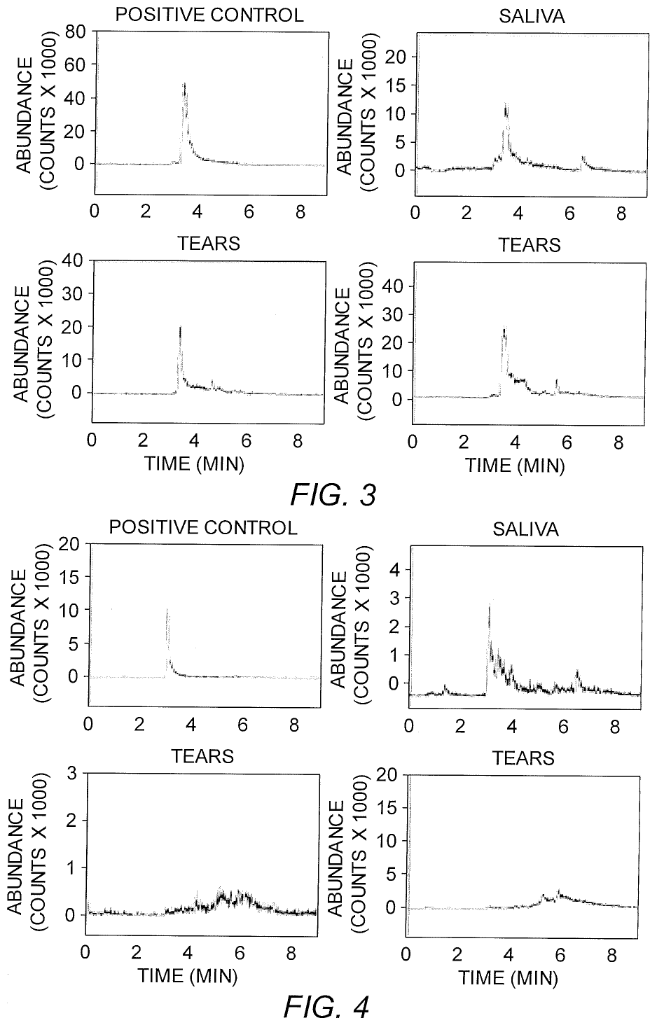

MRM testing was also conducted to assess the presence of H3 and H5 in saliva and tears. Targeted MRM detected the presence of H3 (FIG. 3) and H5 (FIG. 4) in solubilized Syn.H5 (positive control), as well as saliva and tear ocular surface washings from two normal patients.

### Example 4: Histatin Peptides Ameliorate Dry Eye Disease in a Murine Model

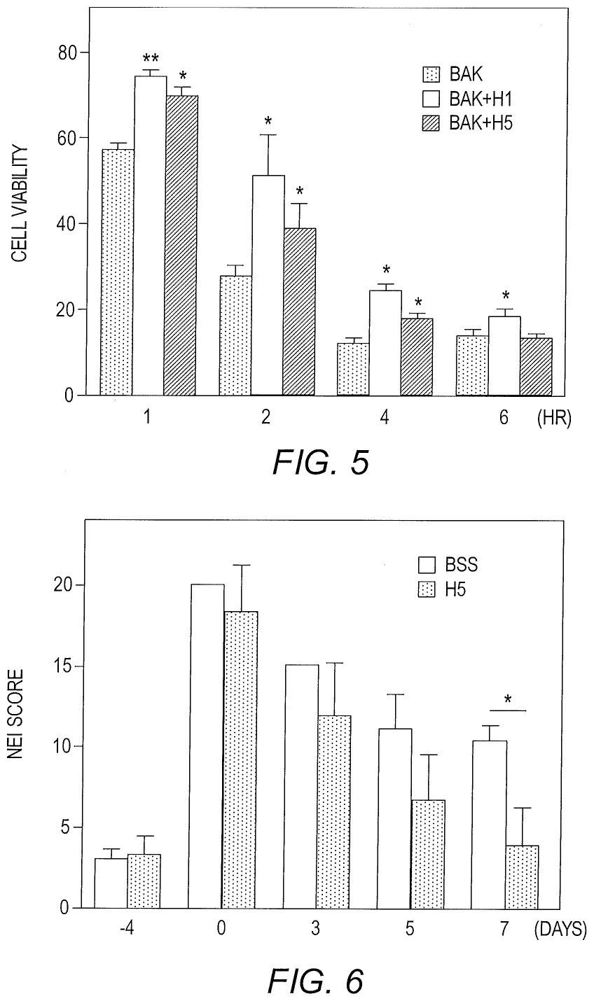

To determine whether histatins could improve symptoms of dry eye disease, cell viability assays were carried out in the presence of the toxic preservative benzalkonium chloride (BAK)(Yang, et al. (2017) Int. J. Mol. Sci. 18(3):509). Human corneal epithelial (HCE) cells were cultivated under standard conditions and viability was assessed using WST-1 reagent, a tetrazolium salt that is reduced to a red formazan dye by the mitochondrial dehydrogenases of metabolically active cells (Bartok, et al. (2015) EXCLI J. 14:123-132). The results of this analysis indicated that HCE viability was improved when BAK-treated cells were co-treated with H1 or H5 (FIG. 5). The level of mRNA and protein expression of dry eye disease-relevant cytokines induced by BAK application to HCE was also significantly reduced when HCE cells were co-treated with H1 or H5 (Table 6).

TABLE 6

| Histatin Co- | Decrease in cytokine expression | | | |
|---|---|---|---|---|
| treatment | IL6 mRNA | IL6 protein | IL8 mRNA | TNFα protein |
| H1 | 82%** | 48%* | 86%** | 12% |
| H5 | 45%* | 45% | 46%* | 32%** |

*<0.05,
**<0.01.
All experiments were performed in triplicate.

To demonstrate the use of histatins in the treatment of dry eye disease, an established murine model was used, i.e., exposure to the benzalkonium chloride (Yang, et al. (2017) Int. J. Mol. Sci. 18(3):509). The results of this analysis indicated that subsequent treatment with twice a day topical H5 (80 μM) reduced standardized dry eye disease score (NEI score) in a statistically significant manner by 7 days (n=5 per condition), as compared to vehicle control (Balanced Salt Solution (BSS))(FIG. 6).

What is claimed is:

1. A method for diagnosing a dry eye disease comprising
(a) determining in a biological fluid sample from a subject a level of one or more histatin peptides and
(b) comparing said level to a reference level,
wherein a reduced level of the one or more histatin peptides as compared to the reference level indicates that the subject has a dry eye disease.

2. The method of claim 1, wherein the dry eye disease is aqueous deficient dry eye (ADDE) disease.

3. The method of claim 1, wherein the one or more histatins are selected from the group of histatin 1, histatin 2, histatin 3, histatin 4, histatin 5, histatin 6, histatin 7, histatin 8, histatin 9, histatin 10, histatin 11 and histatin 12.

4. The method of claim 1, wherein the level of the one or more histatin peptides is reduced by at least 2-fold compared to the reference level.

5. The method of claim 1, wherein the level of the one or more histatin peptides is less than 5 μg/ml.

6. The method of claim 1, further comprising performing a test to assess the subject's tear film quality or ocular surface health.

7. The method of claim 6, wherein tear quality is assessed by measuring tear osmolarity, tear production, tear break-up time, or a combination thereof.

8. The method of claim 6, wherein ocular surface health is assessed by corneal fluorescein staining, Ocular Surface Disease Index scoring, Meiboscale scoring, Dry Eye Questionnaire scoring, or a combination thereof.

9. The method of claim 1, further comprising administering to the subject having a dry eye disease one or a combination of an anti-inflammatory agent, an immunosuppressive agent, a glucocorticoid, a cytostatic agent, an alkylating agent, an antimetabolic agent, a cytotoxic antibiotic, an opioid or a histatin.

10. A method for selecting and treating a subject for a dry eye disease, comprising:
(a) determining in a biological fluid sample from a subject a level of one or more histatin peptides;
(b) comparing said level to a reference level; and
(c) treating the subject with one or a combination of an anti-inflammatory agent, an immunosuppressive agent, a glucocorticoid, a cytostatic agent, an alkylating agent, an antimetabolic agent, a cytotoxic antibiotic, an opioid or a histatin when the level of the one or more histatin peptides are reduced in biological fluid sample from the subject compared to the reference level.

11. The method of claim 10, wherein the dry eye disease is aqueous deficient dry eye (ADDE) disease.

12. The method of claim 10, wherein the one or more histatins are selected from the group of histatin 1, histatin 2, histatin 3, histatin 4, histatin 5, histatin 6, histatin 7, histatin 8, histatin 9, histatin 10, histatin 11 and histatin 12.

13. The method of claim 10, wherein the level of the one or more histatin peptides is reduced by at least 2-fold compared to the reference level.

14. The method of claim 10, wherein the level of the one or more histatin peptides is less than 5 μg/ml.

15. A method for treating a dry eye disease or other ocular disease comprising administering to a subject having a dry eye disease or other ocular disease an effective amount of one or more histatins thereby treating the subject's dry eye disease or other ocular disease.

16. The method of claim 15, wherein the one or more histatins are selected from the group of histatin 1, histatin 2, histatin 3, histatin 4, histatin 5, histatin 6, histatin 7, histatin 8, histatin 9, histatin 10, histatin 11 and histatin 12.

17. The method of claim 15, wherein the other ocular disease is ocular surface disease, ocular or intraocular inflammation, ocular wounding, ocular surface wounding, ocular epithelial dysfunction or damage, intraocular or ocular surface malignancy.

18. A kit for diagnosing dry eye disease comprising
(a) a device for collecting a biological fluid sample, and
(b) an antibody that selectively binds histatin 1, an antibody that selectively binds histatin 2, an antibody that selectively binds histatin 3, an antibody that selectively binds histatin 4, an antibody that selectively binds histatin 5, an antibody that selectively binds histatin 6, an antibody that selectively binds histatin 7, an antibody that selectively binds histatin 8, an antibody that selectively binds histatin 9, an antibody that selectively binds histatin 10, an antibody that selectively binds histatin 11, an antibody that selectively binds histatin 12, or a combination thereof.

19. The kit of claim 18, further comprising a test to assess the subject's tear film quality or ocular surface health.
